(19) United States
(12) Patent Application Publication
(10) Pub. No.: US 2024/0363210 A1
(43) Pub. Date: Oct. 31, 2024

(54) METHODS AND SYSTEMS FOR STRUCTURING MEDICAL REPORT TEXTS

(71) Applicant: Smart Reporting GmbH, Munich (DE)

(72) Inventors: Isabel Cristina PÉREZ VERONA, Ismaning (DE); Michael STROBEL, Munich (DE); Wieland SOMMER, Munich (DE)

(21) Appl. No.: 18/556,719

(22) PCT Filed: Apr. 22, 2021
(86) PCT No.: PCT/EP2021/060587

§ 371 Date: Oct. 23, 2023

(51) Int. Cl. G16H 15/00; G06F 40/205; G06F 40/30

(52) U.S. Cl. CPC G16H 15/00; G06F 40/205; G06F 40/30

(57) ABSTRACT

Methods, systems, and computer program products for structuring medical report texts are provided. In a method, text is received and parsed to obtain a plurality of data elements, connected by semantic connectors. Based thereon, a graph of annotated data elements is generated. In particular, each of the obtained data elements is annotated using medical knowledge bases, which are indicative of medical vocabulary and/or medical ontology and/or medical statistics. To generate a graph, relationships are established between the annotated data elements. The generated graph is embedded into one of a plurality of target structures, wherein each of the target structures has an order criterion. By the embedding, a structured text is provided, which comprises the annotated data elements ordered according to the order criterion of the one of the plurality of target structures, into which the generated graph has been embedded.

### FIELD OF THE INVENTION

This invention relates to the field of creating structured medical report texts.

### BACKGROUND OF THE INVENTION

When drafting a medical report, e.g. of a patient diagnosis, physicians summarize their observations made and the conclusions drawn as well as treatment to be followed.

In many cases, reports are free text offering the reporting physician a high degree of freedom to follow their personal preferences. As a result, the style, wording, and layout differ from physician to physician as well as from case to case. Such reports are typically not machine-readable, not standardized, and not analyzable. Moreover, they are prone to artifacts, and they might be ambiguous or incomplete. Such free-form medical reports are not readily accessible for any type of downstream analysis.

To overcome the drawbacks of free-text reports, so-called structured reports were introduced. For instance, a structured report may be machine-readable, may have an at least partially standardized structure and may contain the same elements, wording, and layout as other comparable structured reports.

Various approaches have been suggested to create structured report. Conventionally, pre-generated report templates can be used, which provide case-specific structure and include recommended reporting steps. Alternatively, a user may select predefined elements, which are then combined to form a report. However, such procedures are perceived by many users as not flexible enough to adapt to peculiarities of a given medical case.

Other approaches serve to create a structured report from a dictated audio file. U.S. Pat. No. 7,958,443 B2 describes such an approach. When dictating the report, a doctor uses predefined key words as section headings. After speech recognition, the speech-recognized text is searched for the predefined key words and a computer automatically locates and marks each heading in the speech-recognized text file. However, such approaches do not provide a high degree of structure, but merely insert predefined section headings into a free-text.

Against this background, there is a need to improve the generation of structured medical reports.

### SUMMARY OF THE INVENTION

The invention provides methods, systems, and computer program products to improve the generation of structured medical report texts. Aspects of the invention are defined in the independent claims. The dependent claims relate to preferred embodiments.

One aspect of the invention concerns a computer-implemented method for structuring a medical report text.

According to an embodiment of the invention, text is received and parsed to obtain a plurality of data elements, connected by semantic connectors.

Furthermore, a graph of annotated data elements is generated. To this end, each of the obtained data elements is annotated using medical knowledge bases. Each of the medical knowledge bases is indicative of medical vocabulary and/or medical ontology and/or medical statistics. To generate a graph, relationships are established between the annotated data elements. The generated graph comprises the annotated data elements connected by the relationships.

Finally, the generated graph is embedded into one of a plurality of target structures, wherein each of the target structures has an order criterion. By the embedding, a structured text is provided, which comprises the annotated data elements ordered according to the order criterion of the one of the plurality of target structures, into which the generated graph has been embedded.

Another aspect of the invention concerns a computer system for structuring a medical report text. The computer system comprises a memory unit, a text receiving unit, and a processing unit.

The memory unit is configured to store medical knowledge bases and a plurality of target structures. Each of the medical knowledge bases is indicative of medical vocabulary and/or medical ontology and/or medical statistics. Each of the target structures has an order criterion.

The text receiving unit is configured to receive text. The processing unit is configured to parse the text received by the text receiving unit, in order to obtain a plurality of data elements, connected by semantic connectors.

The processing unit is further configured to generate a graph of annotated data elements. To this end, the processing unit is configured to annotate each of the obtained data elements using the medical knowledge bases, and to establish relationships between the annotated data elements such that a graph is generated. The generated graph comprises the annotated data elements connected by the relationships.

Finally, the processing unit is configured to embed the generated graph into one of the plurality of target structures to provide a structured text. The structured text comprises the annotated data elements, ordered according to the order criterion of the one of the plurality of target structures, into which the generated graph has been embedded.

Another aspect of the invention concerns a computer-program product for structuring a medical report text according to independent claim 15.

The above and other advantages result from the following considerations, where aspects and embodiments of the invention are discussed and, where appropriate, reference is made to the corresponding drawings, which show preferred embodiments of the invention for illustration purposes. However, these embodiments do not necessarily represent the full scope of the invention.

### BRIEF DESCRIPTION OF THE DRAWINGS

FIG. 1 shows a schematic illustration of the steps of a method according to the invention;

FIG. 2 shows a schematic illustration of an example of an excerpt of a knowledge base for use in the invention;

FIG. 3 shows a schematic illustration of an example of a target structure for use in the invention;

FIG. 4 shows a schematic representation of an example of a plurality of target structures for use in the invention; and

FIG. 5 shows a schematic illustration of a system according to the invention.

### DETAILED DESCRIPTION

The order of any of the steps described in the following can be changed where appropriate. Furthermore, the steps can be performed individually or in combination with each other, where appropriate. All the method steps described can be performed on a computer system.

One aspect of the invention concerns a computer-implemented method for structuring a medical report text.

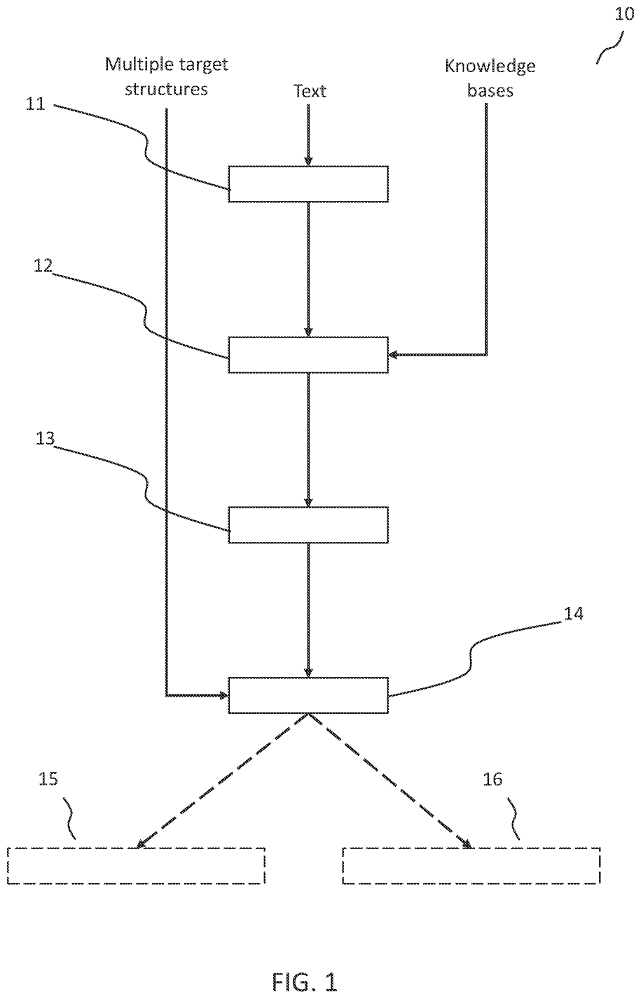

FIG. 1 shows an embodiment of a method 10 according to said aspect. According to the embodiment illustrated in FIG. 1, text is received and parsed in a step 11.

For instance, text may be received from a text input interface, into which a user has typed (or is in the process of typing) a medical report text. Such text input interface may be displayed on a display device and may be manipulated by virtue of an input device, such as a keyboard, mouse or stylus. In some examples, the display device may be a touch-sensitive display device and may serve as both, the display device and the input device.

Additionally or alternatively, text may be received by pre-processing other data, such as by speech-recognizing audio data. In such examples, the user may dictate the text into an audio recording device (e.g. a dictating device or a microphone) and the recorded audio data may be subjected to a speech recognition algorithm to receive (written) text.

In other examples, the pre-processing may comprise image-processing of picture data. For instance, the user may make a handwritten note, which may be scanned or photographed using a picture recording device (e.g. a camera or a scanner) and the recorded image data may be subjected to a handwriting recognition algorithm to receive written text in digital or machine-readable format.

In still other examples, the pre-processing may comprise processing of video data, which contains transcription data. In still other examples, the pre-processing may comprise the processing of image data having “alt-text” (alternative text) descriptions, i.e. image data with short textual descriptions of visual contents of an image.

In any case, the text is received at step 11. It is then parsed to obtain a plurality of data elements connected by semantic connectors. Examples of data elements include words or compound expressions (e.g. expressions consisting of multiple words, but used collectively with a specific meaning), numbers, etc. Examples of semantic connectors include spaces, punctuation marks and conjunctions.

By parsing of the text, the string of words may be divided into its constituents. For instance, a free-form text may be divided into a series of words, the words being separated by a space, punctuation marks, etc. Collectively, the non-word constituents (e.g. space, punctuation marks, conjunctions etc.) may be referred to as semantic connectors. Thus, in one example of parsing, a paragraph may be divided into a series of sentences and each sentence may be divided into a series of words and/or compound expressions, connected by semantic connectors.

The parsing into such data elements allows for a fine-grained structuring, as will become apparent throughout the further description. The data elements obtained by the parsing may be used as units of the structuring.

The plurality of data elements obtained at step 11 are then used to generate a graph of annotated data elements, at steps 12 and 13.

At step 12, each of the obtained data elements is annotated using medical knowledge bases. Each of the medical knowledge bases is indicative of medical vocabulary and/or medical ontology and/or medical statistics.

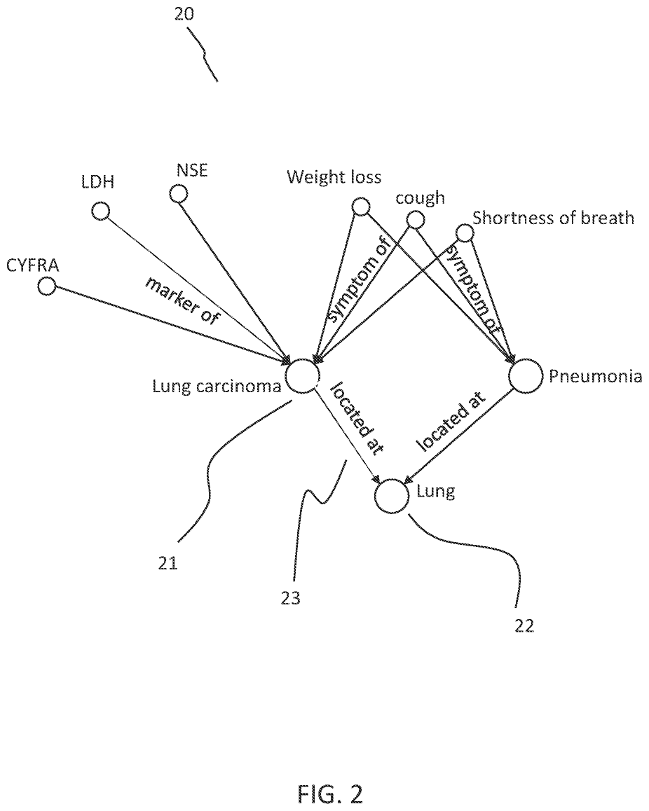

Each of the data elements are thus treated as basic units, which may be associated to knowledge, represented by specific terms existing in one or more knowledge bases. An exemplary knowledge base will be described with reference to FIG. 2.

FIG. 2 shows a schematic illustration of an example of an excerpt of a knowledge base 20. The knowledge base 20 shown is a semantic network. It is graph with vertices and edges. The vertices are shown as circles in FIG. 2. For ease of illustration, two vertices 21, 22 are marked by reference signs. Vertex 21 designates “lung carcinoma”, whereas vertex 22 designates “lung”.

Vertices of the semantic network are connected by edges, which are shown as straight lines/arrows in FIG. 2. For ease of illustration, edge 23 connecting vertices 21, 22 is marked by a reference sign. An edge between two vertices describes a relation between the concepts designated by the respective vertices. For instance, the relation “located at” describes the relation between “lung carcinoma” and “lung”, since “lung carcinoma” is “located at” the “lung”. The relation “located at” is directed, as can be seen from the arrow directed towards vertex 22. (This means that “lung carcinoma is located at the lung”, but the relation shall not be read the other way in the sense of “lung is located at the lung carcinoma”.) The knowledge base 20 shows further relations: For instance, “CYFRA” (short for: cytokeratin 19 fragment), “LDH” (short for: lactate dehydrogenase) and “NSE” (short for: neuron specific enolase) are markers of lung carcinoma.

Further relations include that “weight loss”, “cough” and “shortness of breath” are “symptoms of” lung carcinoma. At the same time, they are also “symptoms of” another disorder, namely “pneumonia”. The latter is also located at the lung.

In some knowledge bases, vertices may comprise or be associated with additional information, such as properties. For instance, vertex 21 may comprise property information that this vertex (lung carcinoma) relates to information of type “disorder”, whereas vertex 22 may comprise property information that this vertex (lung) relates to information of type “organ”.

Similarly, the vertices relating to “weight loss”, “cough” and “shortness of breath” may be marked as relating to information of type “finding”.

The knowledge base 20, an excerpt of which is shown in FIG. 2, is an illustrative example with few nodes and vertices. For instance, it may be a knowledge base specific to a given medical domain, e.g. pulmonology, or it may be a general knowledge base covering many domains. In any case, the skilled person will understand that more complex and more detailed knowledge bases may be used. For instance, knowledge bases representing standardized medical ontologies, such as SNOMED CT or ICD-10, may be used. Such knowledge bases may include a vast number of entries (nodes) and relations, such as multiple hundred thousands of entries in the current version of SNOMED CT. Furthermore, the content of the knowledge bases to be used may be dynamically updated to reflect the expansion of medical knowledge.

The knowledge base 20 may be implemented in a computer system, for instance, using non-relational databases, such as a graph database.

Returning to FIG. 1, the step 12 of annotating each data element using medical knowledge bases allows to enrich the text (which text may focus on, for instance, a particular disease of a particular patient) with more general knowledge from the medical field. For instance, considering the example of a knowledge base indicative of medical ontology, when the physician uses the compound expression “lung cancer” in the text, it may be annotated being of type “disorder”, whereas the compound expression “shortness of breath” may be annotated as being of type “finding”. Such annotating may be done by extracting/copying the knowledge to the data elements or by adding a link at/from the respective data element to the corresponding node in the knowledge base, e.g. using identifiers for each node of the knowledge bases.

It further allows to annotate the text with synonyms of a term used by the physician. For instance, considering the example of a knowledge base indicative of medical vocabulary in English language, when the physician uses the compound expression “lung cancer” in the text, it may be annotated as being synonymous to “lung carcinoma”.

The particular type of annotation may depend on the knowledge base(s) used. In preferred embodiments, multiple knowledge bases are used for annotation, e.g. with overlapping nodes. Each data element may thus be enriched with one or more annotations. This allows for a more detailed and more diverse annotation.

As mentioned above, the plurality of data elements obtained at step 11 is used at step 12 to obtain annotated data elements and at step 13 to generate a graph of said annotated data elements. In particular, relationships are established between the annotated data elements at step 13. The generated graph comprises the annotated data elements connected by the relationships.

In particular, the relationships established between data elements may be represented as edges of the graph, which connect nodes of the generated graph. Relationships may be established based on various considerations, as will be apparent from the following examples.

In a first example, relationships between annotated data elements may be based at least on the medical knowledge bases, the established relationship being indicative at least of medical knowledge.

This allows to enrich the text to be structured with medical knowledge. Such added structure may for instance relate to knowledge, which some physicians might implicitly read along when reading the free-form text. However, such implicit background knowledge might not be readily available in applications of machine-based data analysis.

For this purpose, one or more of the data elements may for instance be matched to one or more nodes of the medical knowledge bases and edges relating to said one or more nodes are extracted from the knowledge bases and established as relationships of the one or more data elements in the graph to be generated.

Using the illustrative example of knowledge base 20 of FIG. 2, when the text to be structured contains the word “cough” along with the word “pneumonia”, these two data elements may be set as nodes of the graph to be generated at step 13. These two nodes are then connected in the graph to be generated by an edge representing that “cough” is a “symptom of” “pneumonia”, i.e. the directed edge representative of the “symptom of” relationship is transferred from the knowledge base to the text to be structured (by means of the graph to be generated at step 13).

This allows to explicitly link the content of the text to be structured with medical knowledge. In a second example of establishing the relationships between data elements, the text may be used as a basis. In this case, the established relationships are indicative at least of semantic or syntactic relationships between the data elements.

To this end, one or more of the data elements can be set as nodes, whereas semantic connectors (connecting said data elements in the parsed text) are set as edges and established as relationships of said text data elements.

For instance, when the text to be structured (e.g. a medical report dictated by a physician with reference to a particular patient) states that “the value of cytokeratin 19 fragment level is 20 ng/mL”, it may be established that the data element “20 ng/mL” is related to the data element “cytokeratin 19 fragment”, by virtue of a relationship “is value of”. As a result of this establishing, an edge may be added to the graph to be generated between the nodes corresponding to the compound expression “cytokeratin 19 fragment” and the compound expression “20 ng/mL”. The edge may be characterized as representing the relationship “is value of”, directed from “20 ng/mL” to “cytokeratin 19 fragment”.

In some embodiments, the establishing of relationships may comprise both, the first example (i.e. establishing relationships based on medical knowledge of the knowledge bases) and the second example (i.e. establishing relationships based on semantic relations in the input text).

This allows to enrich the free-form input text by taking into consideration both, the general medical knowledge of the knowledge bases and the particulars of the patient's case.

In any case, by virtue of steps 12 and 13, a graph is generated, which comprises the annotated data elements (nodes) connected by relationships (edges).

Finally, at step 14, the generated graph is embedded into one of a plurality of target structures, wherein each of the target structures has an order criterion. By the embedding, a structured text is provided, which comprises the annotated data elements ordered according to the order criterion of the one of the plurality of target structures, into which the generated graph has been embedded.

Examples of target structures with an order criterion include decision trees, ordered graphs, hierarchical graphs. Examples of an order criterion include a hierarchy. To provide a structured text, nodes of the selected target structure can be used e.g. as section headings, or component blocks in the output text.

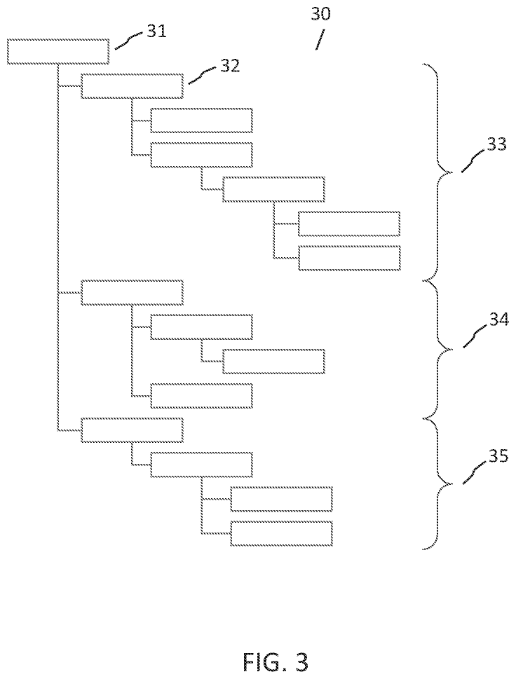

An example of a hierarchical graph will be described with reference to FIG. 3. FIG. 3 schematically shows a hierarchical data structure 30, which in this case is implemented as a tree. The data structure 30 consists of data structure elements 21, which in this example are combined to subgraphs 33, 34 and 35. The modular design has the advantage that other target structures can be quickly created in a modular fashion. For example, by removing module 33, a simpler target structure may be added to the plurality of target structures.

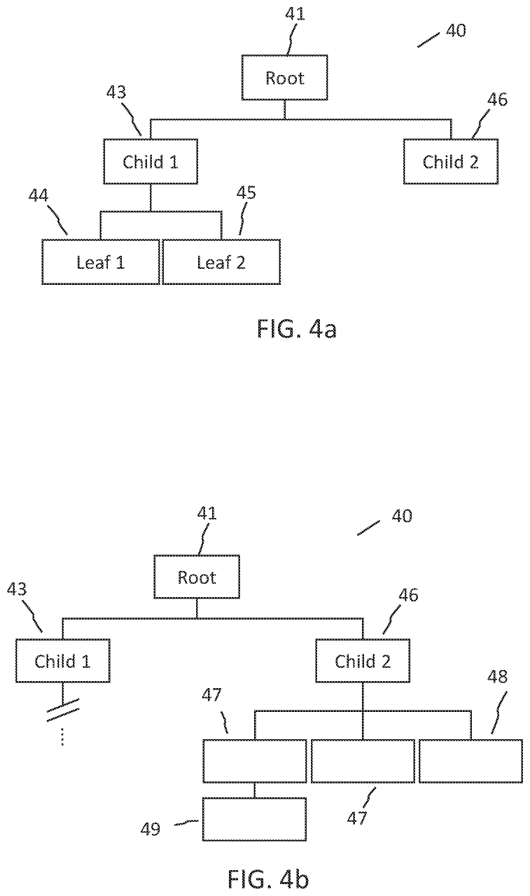

Two further examples of tree data structures are given in FIGS. 4a and 4b. In the following, an illustrative description will be given using the example of tree data structures as target structures. A tree data structure represents a directed rooted tree, i.e. a hierarchical data structure with a root, one or more branch nodes and a plurality of leaves.

In the example of FIG. 4a, the tree 40 comprises a root 41 with two children 43, 46. Child node 43 is a branch node (or internal node), meaning that it has its own children, namely leaf nodes 44, 45. Child 46 on the other hand does not have further children, but is a leaf node itself.

FIG. 4b shows another tree 40, which has a different arrangement. In particular, it features an additional level by virtue of leave 49.

The trees shown in FIGS. 4a and 4b collectively may be referred to as a plurality of target structures. The use of a plurality of target structures also allows for custom-made structures, e.g. reflecting the conventions of a particular field or even of a particular physician, along with more generic structures.

More generally, a plurality of target structures may comprise target structures which differ, e.g. in their size (i.e. number of nodes), breadth (i.e. number of leaves), height (i.e. longest path from the root to any leave, or number of levels), etc.

Returning to FIG. 1, the step 14 of embedding the generated graph may for instance be performed by matching the generated graph to each of the plurality of target structures and selecting one of the plurality of target structures based on the result of the matching. In particular, the one of the plurality of target structures may be selected such that the vertices of the graph (i.e. the graph of annotated data elements) is optimally embedded in the vertices of the selected one of the target structures.

For instance, the matching may be performed by exploring multiple paths across multiple hierarchy levels of a search space tree, in particular using a search tree algorithm.

The embedding is such that the structure of the graph (including the relationships and annotations encoded within the graph, i.e. the added value of steps 12 and 13) are preserved. At the same time, by virtue of the target structure having an order criterion, it is possible to provide a text, as the order criterion allows to put the various data elements into a sequence. For providing the text, the semantic connectors identified at step 11 are used to concatenate the various annotated data elements.

By using a plurality of target structures, a wide variety of reports may be accounted for. For instance, based on the level of detail of the report (i.e. the number of data elements and relationships between the data elements), a target structure with a matching depth may be selected, by virtue of the embedding using an algorithm for optimal embedding. A more detailed report will give rise to a more complex graph and a correspondingly large target structure to be selected, whereas a short report with fewer data elements and fewer relations may give rise to a graph which may be embedded into a smaller target structure. By using a plurality of target structures, the present teaching allows to flexibly accommodate a wide variety of reports. The present teaching thus increases the flexibility of structuring text.

In some embodiments, the method may further comprise one or more steps 15, 16 of outputting the structured text. For instance, at step 15, the text is outputted to a human-readable output as a digital text document. Such outputting may involve an output device, such as a display device or a printer. In particular, it may comprise outputting of the text as such. Additionally, it may also comprise outputting markers indicative of the structure, which has been added by virtue of steps 11 to 14. For instance, to give one illustrative example, the outputting of the term “lung carcinoma” may be highlighted (e.g. by color, font, etc. or other form of highlighting) as pertaining to type “disorder”, whereas “findings” (such as the symptom cough) are highlighted differently.

The output can be navigated using a navigation tool, such as a keyboard/mouse, voice commands, or the control keys of a dictation device.

Additionally or alternatively, at step 16, the text is outputted to a database for machine-based data analysis. For instance, the annotated and structured text may be stored in a database of patient records. The database may then be queried, using particular queries. For instance, in an application of data mining, the database may be queried to output the reports dealing with “lung cancer” and wherein the value of CYFRA is above “18 ng/mL”. Using the reports structured by the method of the present disclosure allows that such queries will also reveal reports where the term “lung cancer” has not been used (but a synonym such as “lung carcinoma”). Further, it allows to reveal reports with a CYFRA level of e.g. 20 ng/mL, since the corresponding data element has been intelligently mapped as referring to a value of CYFRA.

Such teaching may be advantageous in disciplines which involve a high level of transparency within the decision-making process, by ensuring that the input data can be analyzed to extract conclusions.

As shown in FIG. 1, a method 10 may comprise both steps 15 and 16. For instance, during actual dictating/writing of the medical report text, the method 10 may be carried out in real-time, such that the text is concurrently outputted, at step 15, to a display device for review by the physician. At the same time, it may be outputted, at step 16, to a machine-based data analysis for retrieval of decision support data. The decision support data may then also be outputted to the physician, e.g. as a recommendation of potential treatments, e.g. based on a collection of reports with similar characteristics.

More generally, applications of the reports structured according to the present disclosure include medical documentation, health treatment analysis (HTA), treatment effectiveness, transfer to registries or authorities, development of decision support systems. It allows for an efficient machine-based analysis work-flow, even if it was collected in free-text format. At the same time, the perceived freedom for the physician of writing/dictating free-form text is maintained.

Another aspect of the invention concerns a computer system for structuring a medical report text.

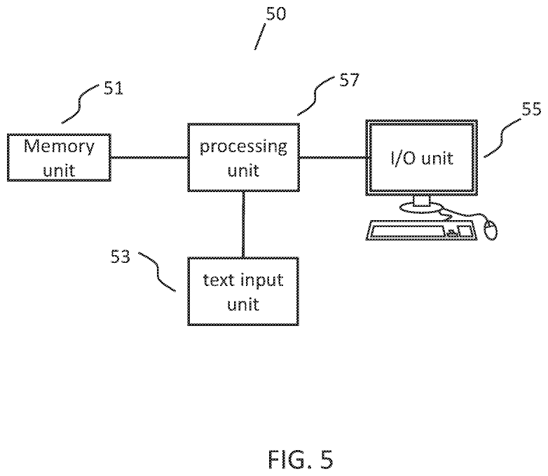

FIG. 5 illustrates such a system 50 comprising a memory unit 51, a text input unit 53 and a processing unit 57.

The memory unit 51 is configured to store medical knowledge bases and a plurality of target structures. In some examples, the memory unit may be implemented as a single device. In other examples, the storing of medical knowledge bases and the storing of plurality of target structures may be functionally separated, e.g. to allow for separately updating the knowledge bases and for updating the plurality of target structures. For ease of illustration, in FIG. 5, a single memory unit is shown, which stores the medical knowledge bases as well as the plurality of target structures.

The text receiving unit 53 is configured to receive text, for instance from a typing device (keyboard, touch screen, etc.), from an audio recording device (microphone, dictating device, etc.) or from a picture recording device (camera, scanner). The processing unit 57 is configured to parse the text received by the text receiving unit, to obtain a plurality of data elements, connected by semantic connectors.

The processing unit 57 is further configured to generate a graph of annotated data elements, essentially as described with reference to steps 12 and 13 of FIG. 1. To this end, the processing unit 57 is configured to annotate each of the obtained data elements using the medical knowledge bases of memory unit 51, and to establish relationships between the annotated data elements such that a graph is generated. The generated graph comprises the annotated data elements connected by the relationships.

Finally, the processing unit 57 is configured to embed the generated graph into one of the plurality of target structures of memory unit 51, to provide a structured text. The structured text comprises the annotated data elements, ordered according to the order criterion of the one of the plurality of target structures, into which the generated graph has been embedded.

In some embodiments, system 50 further comprises an output unit 55 configured to display or otherwise output the structured text and, if required, also a text input interface for facilitating input of text using the text input unit. The processing unit 57 can further be configured to control the output unit 55 and the text input unit 52 and to carry out any of the method steps described above.

The processing unit 57 can, e.g., consist of a single- or multi-core processor or multiple physical processors (also referred to as multiple sockets).

According to another aspect of the invention, a computer program product for creating structured medical findings reports is provided, which is stored on a non-volatile storage medium and contains computer-readable instructions for carrying out any of the method steps described above. In particular, the computer program product may be executed using the system 50.

Using the aspects and embodiments of this invention, the established efficient way of working with medical report text (such as working with free text via keyboard input or dictation) is maintained and at the same time medical report texts are structured and recorded in a data structure.

Medical data becomes machine-readable and machine-interpretable due to the structured acquisition and can be used directly for analytical purposes. This makes the data digitally available to other IT applications (e.g., controlling, artificial intelligence, etc.). Thus, important expert information, entered as free text, is not lost. In addition, there is the possibility of annotating and structuring the data and thus linking the data elements with unique meaningful relationships. The approach taught herein thus combines fine granularity of the structure with a high flexibility of accommodating a wide variety of reports.

What is claimed is:

1. A computer-implemented method for structuring a medical report text comprising:
receiving text and parsing the received text to obtain a plurality of data elements, connected by semantic connectors;
generating a graph of annotated data elements, the generating comprising:
annotating each of the obtained data elements using medical knowledge bases, each of the medical knowledge bases being indicative of medical vocabulary or medical ontology or medical statistics; and
establishing relationships between the annotated data elements such that a graph is generated, the graph comprising the annotated data elements connected by the relationships;
embedding the generated graph into one of a plurality of target structures, each of the target structures having an order criterion, to provide a structured text comprising the annotated data elements ordered according to the order criterion of the one of the plurality of target structures, into which the generated graph has been embedded.

2. The method according to claim 1, further comprising outputting the structured text, wherein the outputting is to a human-readable output of an output device or to a database for machine-based data analysis.

3. The method according to claim 1, wherein the established relationships between data elements are represented as edges between nodes of the generated graph.

4. The method according to claim 1, wherein establishing relationships between annotated data elements is based at least on the medical knowledge bases, the established relationship being indicative at least of medical knowledge.

5. The method according to claim 4, wherein, in the establishing relationships between annotated data elements, one or more of the data elements are matched to one or more nodes of the medical knowledge bases and edges relating to said one or more nodes are extracted and established as relationships of the one or more data elements.

6. The method according to claim 1, wherein the establishing relationships between annotated data elements is based at least on the received text, the established relationship being indicative at least of semantic relationships between the data elements.

7. The method according to claim 6, wherein, in the establishing relationships between annotated data elements, one or more of the data elements are set as nodes and semantic connectors, which connect the data elements in the parsed text, are set as edges and established as relationships of the one or more data elements.

8. The method according to claim 1, further comprising performing a pre-processing of at least one of the following, such that text is received by:
speech-recognizing audio data;
image-processing picture data; or
transcription-processing of video data.

9. The method according to claim 1, wherein the plurality of target structures are selected from one or more of decision trees, ordered graphs, or hierarchical graphs.

10. The method according to claim 1, wherein embedding of the generated graph comprises matching the generated graph to each of the target structures and selecting one of the plurality of target structures based on the result of the matching.

11. The method according to claim 10, wherein the matching comprises exploring multiple paths or multiple hierarchy levels of a search space tree using a search tree algorithm.

12. The method according to claim 1, wherein the medical knowledge bases comprise semantic networks.

13. A computer system for structuring a medical report text, comprising
a memory unit configured to store medical knowledge bases, each of the medical knowledge bases being indicative of at least one of medical vocabulary, medical ontology, or medical statistics, and to store a plurality of target structures, each of the target structures having an order criterion;
a text receiving unit configured to receive text; and
a processing unit, configured to:
parse the text received by the text receiving unit, to obtain a plurality of data elements, connected by semantic connectors;
generate a graph of annotated data elements, wherein the generating comprises:
annotating each of the obtained data elements using the medical knowledge bases; and
establishing relationships between the annotated data elements such that a graph is generated, the graph comprising the annotated data elements connected by the relationships;
embed the generated graph into one of the plurality of target structures, to provide a structured text comprising the annotated data elements ordered according to the order criterion of the one of the plurality of target structures, into which the generated graph has been embedded.

14. The computer system according to claim 13, further comprising an output unit configured to output the structured text, and a text input unit configured to receive user input, wherein the processing unit is further configured to carry out a method including steps a comprising:
receiving text and parsing the received text to obtain a plurality of data elements, connected by semantic connectors:
generating a graph of annotated data elements, the generating comprising:
annotating each of the obtained data elements using medical knowledge bases, each of the medical knowledge bases being indicative of medical vocabulary or medical ontology or medical statistics; and
establishing relationships between the annotated data elements such that a graph is generated, the graph comprising the annotated data elements connected by the relationships:
embedding the generated graph into one of a plurality of target structures, each of the target structures having an order criterion, to provide a structured text comprising the annotated data elements ordered according to the order criterion of the one of the plurality of target structures, into which the generated graph has been embedded.

15. A computer program product for structuring a medical report text, which is stored on a non-volatile storage medium and contains computer-readable instructions for carrying out steps of a method a including steps comprising:
receiving text and parsing the received text to obtain a plurality of data elements, connected by semantic connectors:
generating a graph of annotated data elements, the generating comprising:
annotating each of the obtained data elements using medical knowledge bases, each of the medical knowledge bases being indicative of medical vocabulary or medical ontology or medical statistics; and
establishing relationships between the annotated data elements such that a graph is generated, the graph comprising the annotated data elements connected by the relationships;
embedding the generated graph into one of a plurality of target structures, each of the target structures having an order criterion, to provide a structured text comprising the annotated data elements ordered according to the order criterion of the one of the plurality of target structures, into which the generated graph has been embedded.
